(19) United States
(12) Patent Application Publication
(10) Pub. No.: US 2026/0191463 A1
(43) Pub. Date: Jul. 9, 2026

(54) SYSTEMS AND METHODS FOR TRAINING MACHINE LEARNING MODELS FOR USE IN A PLURALITY OF SENSORY MONITORING DEVICES

(71) Applicant: MEDASENSE BIOMETRICS LTD., Ramat Gan (IL)

(72) Inventors: Galit ZUCKERMAN-STARK, Tel Aviv (IL); Omer Miller ROTEM, Ramat Gan (IL)

(21) Appl. No.: 19/419,216

(22) Filed: Dec. 15, 2025

Related U.S. Application Data
(60) Provisional application No. 63/742,958, filed on Jan. 8, 2025.

(51) Int. Cl. A61B 5/00; G16H 40/20

(52) U.S. Cl. CPC A61B 5/4824; G16H 40/20

(57) ABSTRACT

Methods and systems for receiving, by at least one processor, for each of a plurality of subjects at defined time intervals during a medical procedure, a subject-specific sensory metric output and a subject-specific sensor dataset. The subject-specific sensory metric output is outputted by a first sensory-monitoring device (SMD) of a first SMD type. The subject-specific sensor dataset is outputted by sensors monitoring a body of each subject from the plurality of subjects, and to be utilized by a second SMD of a second SMD type. The at least one processor executes code to correct for data collection deficiencies, to generate a training dataset for training a machine learning model (MLM) based on the subject-specific sensory metric output and a corrected subject-specific sensor dataset, to train the MLM, and to provide the trained MLM to the second SMD to facilitate medical actions during subsequent medical procedures.

### FIELD OF TECHNOLOGY

The present disclosure relates to the field of medical technology and artificial intelligence, specifically to systems and methods for training machine learning models for use in a plurality of sensory monitoring devices.

### BACKGROUND OF TECHNOLOGY

Pain is an unpleasant sensory and emotional experience associated with actual or potential tissue damage. Traditional methods of pain assessment rely on subjective patient feedback, which can be unreliable, especially in cases where patients are unable to communicate effectively. There is a need in the art for objective, reliable, and accurate methods to assess pain that can be used across various patient demographics and conditions.

### SUMMARY

The present disclosure provides systems and methods for training a machine learning model for use in a sensory-monitoring device.

In some embodiments, the present disclosure provides an exemplary technically improved computer-based method that may include at least the following steps of interacting, by at least one processor, with: at least one first sensory-monitoring device (SMD) of at least one first SMD type to receive, for each of a plurality of subjects at each of a plurality of defined time intervals during a subject-specific medical procedure, at least one subject-specific sensory metric output from at least one first SMD, and at least one data processing interface module to receive, for each of the plurality of subjects at each of the plurality of defined time intervals during the subject-specific medical procedure, at least one subject-specific sensor dataset for each subject outputted by at least one sensor monitoring a body of each subject from the plurality of subjects; where the at least one sensor is to be utilized by at least one second SMD of at least one second SMD type during a subsequent medical procedure; where the at least one subject-specific sensory metric output may be indicative of a level of a sensory event experienced by each subject at each of the plurality of defined time intervals during the subject-specific medical procedure; iteratively identifying, by the at least one processor, at least one first dataset collection deficiency in the at least one subject-specific sensory metric output; iteratively identifying, by the at least one processor, at least one second dataset collection deficiency in the at least one subject-specific sensor dataset; iteratively correcting, by the at least one processor, for the at least one first dataset collection deficiency in the at least one subject-specific sensory metric output, to obtain at least one corrected subject-specific sensory metric output; iteratively correcting, by the at least one processor, for the at least one second dataset collection deficiency in the at least one subject-specific sensor dataset, to obtain at least one corrected subject-specific sensor dataset; generating, by the at least one processor, at least one training dataset for training at least one second sensory-monitoring machine learning model based on: the at least one corrected subject-specific sensory metric output from the at least one first SMD for each of the plurality of subjects at each of the plurality of defined time intervals during the subject-specific medical procedure, and the at least one corrected subject-specific sensor dataset from the at least one sensor to be utilized by the at least one second SMD; training, by the at least one processor, the at least one second sensory-monitoring machine learning model with the at least one training dataset to obtain at least one trained second sensory-monitoring machine learning model; and providing, by the at least one processor, the at least one trained second sensory-monitoring machine learning model to the at least one second SMD of the at least one second SMD type so as to cause at least one medical action during a subsequent subject-specific medical procedure responsive to a subsequent sensory metric output prediction generated in real time by execution of the at least one trained second sensory-monitoring machine learning model by the at least one second SMD.

In some embodiments, the present disclosure provides an exemplary technically improved computer-based system that includes at least the following components of at least one processor and at least one non-transitory memory. The at least one processor may be configured to execute computer code that causes the at least one processor to: interact with: at least one first sensory-monitoring device (SMD) of at least one first SMD type to receive, for each of a plurality of subjects at each of a plurality of defined time intervals during a subject-specific medical procedure, at least one subject-specific sensory metric output from at least one first SMD, and at least one data processing interface module to receive, for each of the plurality of subjects at each of the plurality of defined time intervals during the subject-specific medical procedure, at least one subject-specific sensor dataset for each subject outputted by at least one sensor monitoring a body of each subject from the plurality of subjects. The at least one sensor is to be utilized by at least one second SMD of at least one second SMD type during a subsequent medical procedure. The at least one subject-specific sensory metric output may be indicative of a level of a sensory event experienced by each subject at each of the plurality of defined time intervals during the subject-specific medical procedure. The at least one processor may be configured to iteratively identify at least one first dataset collection deficiency in the at least one subject-specific sensory metric output, iteratively identify at least one second dataset collection deficiency in the at least one subject-specific sensor dataset, iteratively correct for the at least one first dataset collection deficiency in the at least one subject-specific sensory metric output, to obtain at least one corrected subject-specific sensory metric output, iteratively correct for the at least one second dataset collection deficiency in the at least one subject-specific sensor dataset, to obtain at least one corrected subject-specific sensor dataset, generate at least one training dataset for training at least one second sensory-monitoring machine learning model based on: the at least one corrected subject-specific sensory metric output from the at least one first SMD for each of the plurality of subjects at each of the plurality of defined time intervals during the subject-specific medical procedure, and the at least one corrected subject-specific sensor dataset from the at least one sensor to be utilized by the at least one second SMD. The at least one processor may be configured to train the at least one second sensory-monitoring machine learning model with the at least one training dataset to obtain at least one trained second sensory-monitoring machine learning model and provide the at least one trained second sensory-monitoring machine learning model to the at least one second SMD of the at least one second SMD type so as to cause at least one medical action during a subsequent subject-specific medical procedure responsive to a subsequent sensory metric output prediction generated in real time by execution of the at least one trained second sensory-monitoring machine learning model by the at least one second SMD.

Similarly, in some embodiments, the at least one first SMD and the at least one second SMD may be at least one first pain-monitoring device (PMD) and at least one second PMD. In this case, the subject-specific sensory metric output may be a subject-specific pain metric output, indicative of a level of pain experienced by each subject, that may be obtained by the at least one first PMD using at least one PMD sensor monitoring the body of each subject.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 is a schematic block diagram of a system for training a machine learning model for use in a sensory monitoring device in accordance with one or more embodiments of the present disclosure;

FIG. 2 is a schematic block diagram of a sensory monitoring device implemented as a pain monitoring device in accordance with one or more embodiments of the present disclosure;

FIGS. 3A-C are optional depictions of a machine learning module in a processing module in at least one first pain-monitoring device in accordance with one or more embodiments of the present disclosure; and

FIG. 4 is a flowchart of a method for training a machine learning model for use in a sensory monitoring device in accordance with one or more embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “dynamically” and term “automatically,” and their logical and/or linguistic relatives and/or derivatives, mean that certain events and/or actions can be triggered and/or occur without any human intervention.

As used herein, the term “runtime” corresponds to any behavior that is dynamically determined during an execution of a software application or at least a portion of software application.

Machine learning (ML) and Artificial Intelligent (AI) systems may be built around a statistical model trained for a specific task based on past observations, that can predict, or estimate the most probable outcome for a given input. During the training process, pairs of input and output features in data training sets may be fed into the model that provides the output or outcome for a given input. As such, during the training process, the machine-learning model parameters may be iteratively modified so as to cause the input features to map into output features, or outcomes within a predefined predictive accuracy. Generally speaking, the larger the data training set, or the more input/output pairs that may be provided during the training process, the more accurate and robust may be the predictive accuracy of the trained model in mapping a plurality of input features\signals into an output outcome.

Traditional machine learning models may require manual marking of the output in preparation for the training process. In the field of the current disclosure, training a statical model may need expert annotations of nociceptive or painful stimuli. The burden of manual processes and the need for expert knowledge (e.g., physicians that may be experts in pain treatment or anesthesia) may limit the amount of data available for training. This may result in ML systems that may be limited in their potential accuracy.

At least some embodiments of the present disclosure provide novel systems and methods for training AI models, algorithms, or both for new sensory monitoring devices (denoted herein as “second sensory monitoring devices”) using data from previous sensory monitoring devices (denoted herein as “first sensory monitoring devices”) to provide reference/ground-truth data for training datasets. That is, the output of the first sensory monitoring device used as the reference/ground-truth output data may be used to generate a training dataset for training a machine learning model used in a second sensory monitoring device.

In some embodiments, at least one first sensory monitoring device (SMD) of at least one first SMD type may have a first set of at least one sensor used for monitoring a body of a subject during a subject-specific medical procedure. The first sensor output data from the first set of the at least one first sensor may be coupled to the first sensory monitoring device which is used to generate a subject-specific sensory metric output. During the subject-specific medical procedure, a second set of at least one second sensor may be used to monitor the body of the subject. The second sensor output data from the second set of the at least one second sensor may be measured via a data processing interface module and stored in a storage device. The second sensor output data from the second set of the at least one second sensor may be utilized by at least one second SMD of at least one second SMD type during a subsequent medical procedure.

In some embodiments, the second set of the at least one second sensor may be different than the first set of the at least one first sensor.

In some embodiments, the second set of the at least one second sensor may be the same as the first set of the at least one first sensor.

In some embodiments, collecting subject-specific data, e.g., the subject-specific sensory metric output from the at least one first SMD and the second sensor output data to be utilized by the at least one second SMD in the manner described above, over a plurality of subjects may be used to generate at least one training data set for training a machine learning model used by the at least one second SMD of the at least one second SMD type.

In some embodiments, during a subject-specific medical procedure using at least one sensory monitoring device (SMD), two sets of sensors may monitor a body of each subject from the plurality of subjects: a first set of at least one SMD sensor, and a second set of sensors for generating the second sensors datasets. The at least one SMD sensor may be coupled to the at least one sensory monitoring device (SMD), which may be used to generate a subject-specific sensory metric output indicative of a level of sensory event that is experienced by each subject from a plurality of subjects.

Note that the at least one first SMD of at least one first SMD type and the at least one second SMD of the least one second SMD type may be, for example, of different device models for a same manufacturer or from different device manufacturers. The at least one first SMD type with the at least one first MLM and the at least one second SMD type with the at least one second MLM may have a different number and/or a different type of sensors monitoring different physiological signals of a subject's body that are used as inputs to the at least one first MLM and the at least one second MLM to both output a second sensory metric output indicative of the level of the sensory event experienced by each subject during the patient-specific medical procedure.

In some embodiments, the at least one second SMD may be the at least one first SMD. the at least one second SMD type may be the at least one first SMD type, or the at least one second SMD type may be different than the at least one first SMD type.

In some embodiments, the at least one second SMD type may be the at least one first SMD type. The at least one second SMD and the at least one first SMD, or the at least one second SMD is different than the at least one first SMD.

In some embodiments, when a subject undergoes a subject-specific medical procedure, the at least one first and second SMD may each generate at least one first and second subject-specific sensory metric output based on a level of a sensory event that the subject may experience during the subject-specific medical procedure.

In some embodiments, the at least one sensory monitoring device described herein may be at least one pain monitoring device. Thus, at least one subject-specific sensory metric output may be at least one subject-specific pain metric based on a level of pain that the subject may experience during the subject-specific medical procedure. The present method may automate the annotation of pain-related data features with corresponding pain levels. This automated annotation process may accelerate the creation of labeled datasets, enabling the efficient training of AI models for pain assessment so as to facilitate building of a large machine learning (ML) models for pain monitoring devices using the output of existing nociception and/or pain monitoring devices as reference data. The integration of commercially available devices to generate data, with AI-based pain monitoring systems enhances scalability, accuracy, and accessibility in pain assessment.

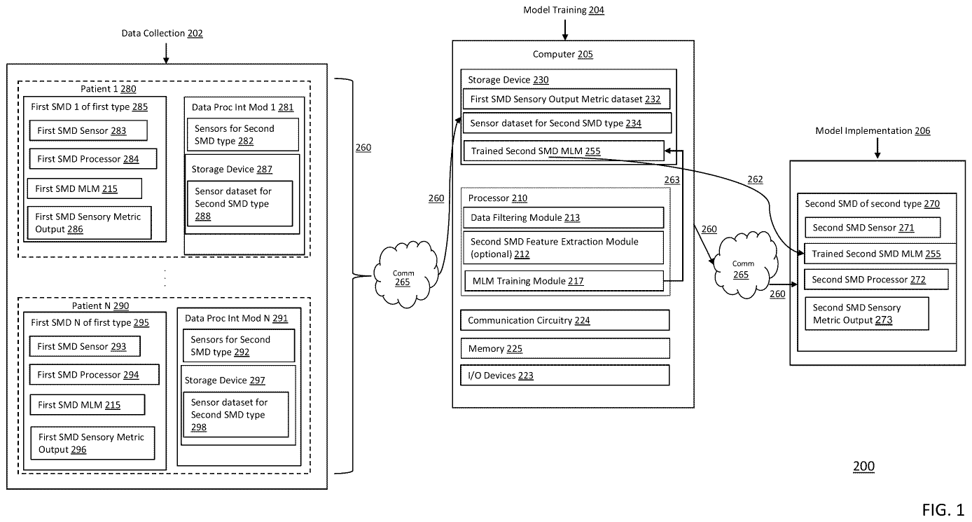

Referring to FIG. 1, a system 200 for training a machine learning model for use in a sensory monitoring device in accordance with one or more embodiments of the present disclosure. The system 200 may facilitate the implementation of three functional subsystems that may include a data collection subsystem 202, a model training subsystem 204, and/or a model implementation subsystem 206.

In some embodiments, the model implementation subsystem 206 may include at least one second SMD 270 of at least one second SMD type. The at least one second SMD 270 may include at least one second SMD sensor 271 and at least one second SMD processor 272. The at least one second SMD processor 272 may be configured to execute a trained second SMD machine learning model (MLM) 255 that may be trained to output the at least one second SMD sensory metric output 273.

In some embodiments, the model training subsystem 204 may be implemented by a computer 205.

In some embodiments, the computer 205 may be communicatively coupled 260 through a communication network 265 to the data collection subsystem 202 and to the model implementation subsystem 206.

In some embodiments, the data collection subsystem 202 may include for a patient 1 280 from the plurality of N patients, where N is an integer: at least one first SMD 1 285 of at least one first SMD type and at least one data processing interface module 1 281. Similarly, the data collection subsystem 202 may include for a patient N 290 from the plurality of N patients: at least one first SMD N 295 of at least one first SMD type and at least one data processing interface module 291.

In some embodiments, the at least one data processing interface module 1 281 and the at least one data processing interface module N 291 respectively may each be at least one recording device.

In some embodiments, the at least one first SMD 1 285 may include at least one first SMD sensor 283, at least one first SMD processor 284 configured to execute at least one first SMD MLM 215 for generating at least one first SMD sensory metric output 286. Similarly, the at least one first SMD N 295 may include at least one first SMD sensor 293, at least one first SMD processor 294 configured to execute at least one first SMD MLM 215 for generating at least one first SMD sensory metric output 296.

In some embodiments, the at least one first sensory metric output 286 from the at least one first SMD 285 of the at least one first SMD type may be used as the reference ground-truth data for training datasets used to train at least one second MLM used in the at least one second SMD of at least one second SMD type that may be different from the at least one first SMD type.

In some embodiments, the at least one data processing interface module 1 281 may include at least one storage device 287 that may store at least one second sensor dataset 288 intended for training any suitable at least one machine learning model to generate the at least one trained second SMD machine learning model (MLM) 255 for the at least one second SMD 270. the at least one data processing interface module N 291 may include at least one storage device 297 that may store at least one sensor dataset 298 intended for training the at least one second SMD machine learning model (MLM) 255 in the at least one second SMD 270.

In some embodiments, the at least one first and second sensory-monitoring device (SMD) 285, 270 may include the at least one first and second SMD processor 284, 272 that may execute computer code to receive sensor data from the at least one first and second SMD sensor 283, 282 which may be inputted to at least one first and second sensory-monitoring decision mechanism module configured to output the at least one first and second subject-specific sensory metric output indicative of the level of the sensory event experienced by the subjects.

In some embodiments, the at least one first sensory-monitoring decision mechanism module may include at least one first sensory monitoring algorithm, at least one first sensory-monitoring machine learning model, or any combination thereof that may be trained by the systems and methods described herein for generating the at least one first subject-specific sensory metric output.

In some embodiments, the at least one second sensory-monitoring decision mechanism module may include at least one second sensory-monitoring algorithm, at least one second sensory-monitoring machine learning model, or any combination thereof that may be trained by the systems and methods described herein for generating the at least one second subject-specific sensory metric output.

In some embodiments in the model training subsystem 204, the computer 205 may include at least one storage device 230, at least one processor 210, at least one non-transitory memory 225, at least one input/output (I/O) device 223, and/or at least one communication circuitry 224. The at least one communication circuitry 224 may facilitate the communication 260 between the computer 205 over the communication network 265 and the data collection submodule 202 and/or the model implementation submodule 206.

Note that the term “patient” and “subject” may be used interchangeably herein.

In some embodiments, the at least one storage device 230, 287, 297 may include for example, but are not limited to a Hard Disk Drive (HDD), a Solid-State Drive (SSD): a flash memory, an Optical Disc Drives (e.g., devices that read and write data on optical discs like CDs, DVDs, and Blu-rays), USB Flash Drive (Small, portable storage devices that connect to a computer via a USB port), an External Hard Drive (e.g., a portable hard drive that can be connected to a computer via USB, Thunderbolt, or other interfaces), and/or a cloud storage service (e.g., Online services that may store data on remote servers. Examples include Google Drive, Dropbox, and OneDrive).

In some embodiments, the at least one processor 210 may execute computer code stored in the at least one non-transitory memory 225 of software modules such as for example, but not limited to a data filtering module 213, a machine learning training module 217, and/or a second SMD feature extraction module 212. The machine learning training module 217 may generate and subsequently store 263 the trained second SMD machine learning model (MLM) 255 in the at least one storage device 230.

In some embodiments, the at least one storage device 230 may store at least one first SMD sensory metric output dataset 232 for each of the N plurality of patients, at least one sensor dataset for the second SMD type 234, and/or the at least one trained second SMD MLM 255. The at least one first SMD sensory metric output dataset 232 is a data collection of the at least one first SMD sensory metric output 286, 296 from each of the N plurality of patients. Similarly, the at least one sensor dataset for the second SMD type 234 is a data collection from the at least one sensor dataset 288, 298 for the second SMD type.

In some embodiments, after the at least one processor using the MLM training module 217 may train the at least one trained second SMD MLM 255, the at least one communication circuitry 224 may communicate 262 the at least one trained second SMD MLM 255 by any suitable method (e.g., USB flash drive, the communication network 265, etc.) to the at least one second SMD 270 of the at least one second SMD type.

In some embodiments, the at least one first SMD sensor 283, 293 and/or the at least one second sensor 282, 292 may include but are not limited to a pressure gauge, a piezoelectric sensor, a camera, an infrared (IR) camera, a thermal imaging camera, a Galvanic Skin Response (GSR) sensor, an Electro Dermal Response (EDR) sensor, a Skin Conductance Response (SCR) sensor, a Electro-Gastro-Gram (EGG) sensor, a Pupil Diameter Measurement (PD) sensor, an Electromyography sensor (EMG), a Frontalis (scalp) Electromyogram (FEMG) sensor, a PhotoPlethysmoGraph (PPG) sensor, an Electro-Cardio-Gram (ECG) sensor, an ElectroEncephaloGraph (EEG) sensor, an ElectroOculaGraph (EOG) sensor, a Blood pressure (BP) sensor, a Laser Doppler Velocimetry (LDV) sensor, a carbon dioxide (CO2) sensor, a Nitrous oxide sensor (N2O), an oxygen (O2) sensor, and/or an Accelerometer.

In some embodiments, the at least one first SMD sensor 283, 293 and/or the at least one second sensor 282, 292 may sense and/or detect and/or monitor physiological signals on a body of a subject that may include, but are not limited to heartrate (HR), heartrate variability metrics (HRV), vaso-constriction\dilation, blood perfusion, Blood oxygen saturation, blood pressure, respiration, internal and/or surface temperature, pupil diameter, GSR, and signals received and/or abstracted and/or derived from ECG, PPG, EOG, EGG, EEG, EMG, EGG, LDV, capnograph and/or accelerometer or any portion or combination thereof. A physiological signal may further include any signal that is measurable and/or detectable from a subject. For example, a camera that measures heart rate (HR) and/or facial expressions, and/or from a sensor that is placed under a mattress on which the subject is laying.

In some embodiments, a training dataset may be generated to train the at least one second MLM 255 to output the at least one sensory metric output for sensor data inputs to the at least one second SMD different from the at least one first SMD sensors. The training dataset may include input and output data features or feature vectors that may respectively include: (1) input sensor data from the at least one second sensor intended to be used in subsequent medical procedures by the at least one second SMD of the at least one second SMD type (e.g., the sensor dataset for the Second SMD type 234) for the plurality of N subjects and (2) the at least one first sensory output metric dataset, or the ground truth output dataset outputted from the at least one first SMD of the at least one first SMD type from the plurality of N subjects.

More details of the sensors, types of sensor data, sensor data features and/or pain metrics may be found in U.S. patent application Ser. No. 15/349,098 filed Nov. 11, 2016, and now U.S. Pat. No. 10,743,778 that issued on Aug. 18, 2020, which is incorporated herein by reference in its entirety.

In some embodiments, a type of sensory event that may be monitored during a medical procedure by at least one sensory monitoring device as in the list hereinbelow may include but is not limited to:
Pain\Nociception monitoring: as disclosed herein,
Autonomous Nervous System (ANS) activity
Vision monitoring: eye tracking devices to measure eye movements, pupil dilation, and/or gaze direction, electroretinography (ERG) that records the electrical response of the retina to light stimulation, optical coherence tomography (OCT) that creates high-resolution images of the retina, electrooculogram (EOG),
Hearing monitoring: Audiometry devices that measure hearing sensitivity at different frequencies, tympanometry devices that evaluate the function of the middle ear, auditory brainstem response (ABR) devices that measure the electrical activity of the auditory nerve and brainstem in response to sound,
Touch monitoring: tactile sensors for measuring pressure, vibration, and/or texture, electrodermal activity (EDA) sensors for measuring skin conductivity, that may be used to assess emotional arousal.
Smell monitoring: electronic nose devices for detecting and/or identify odors using chemical sensors,
Taste monitoring: electronic tongues to detect and analyze taste compounds,
Proprioception monitoring: proprioception sensors to measure the body's sense of position and movement, and
Vestibular monitoring: vestibular sensors to measure balance and spatial orientation.

In some embodiments, the computer 205 may provide 262 the trained second sensory monitoring MLM 255 to the at least one second sensory monitoring device 270 that may include uploading the trained second sensory monitoring MLM 255 to the at least one second sensory monitoring device 270 by any suitable wired and/or wireless communication protocol, and/or from a storage device such as for example but not limited to a USB memory stick.

In some embodiments, the data filtering module 213 may iteratively correct for at least one data collection deficiency in the subject-specific sensor dataset to obtain a corrected subject specific sensor dataset. The at least one data collection deficiency may result in including low quality or erroneous data, which must be handled. Correction of such a deficiency may result in possible loss of data, creating bias offset. Bias offset in a training dataset may further cause systematic errors and/or distortions in the machine learning model predictive performance. This may occur when the training data may not accurately represent the true distribution of the target population due to for example, removing segments of the data, sampling bias or data favoring certain groups or outcomes, labeling bias, and/or measurement bias.

In some embodiments, the at least one dataset collection deficiency in the subject-specific sensor dataset may include deficiencies during the subject-specific medical procedure caused by a patient movement, an interference from other medical equipment, a sensor malfunction, a sensor misplacement on the body, a usage of electric cautery, a usage of diathermy, extreme temperatures, a usage of vasoactive drugs, manipulations that may affect physiological markers, or any combination thereof.

In some embodiments, the data filtering module 213 may iteratively correct the at least one dataset collection deficiency in the subject-specific sensor dataset by iteratively correcting low quality input data, missing input data, or both, in the subject-specific sensor dataset.

In some embodiments, the data filtering module 213 may iteratively correct for similar data collection deficiencies in the at least one first sensory metric output 286, 296 outputted by the at least one first SMD 285, 295.

In some embodiments, the MLM training module 217 may train the second sensory-monitoring machine learning model 255 using the training dataset. The trained second sensory-monitoring machine learning model 255 may then be provided to at least one second sensory-monitoring device 270, which may include at least one second SMD processor 272, to facilitate medical actions during subsequent medical procedures based on sensory metric predictions.

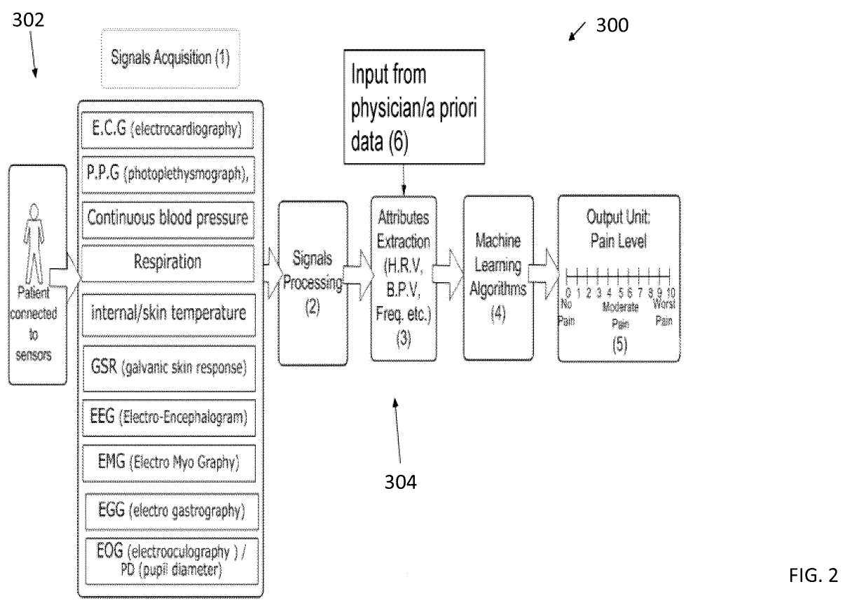

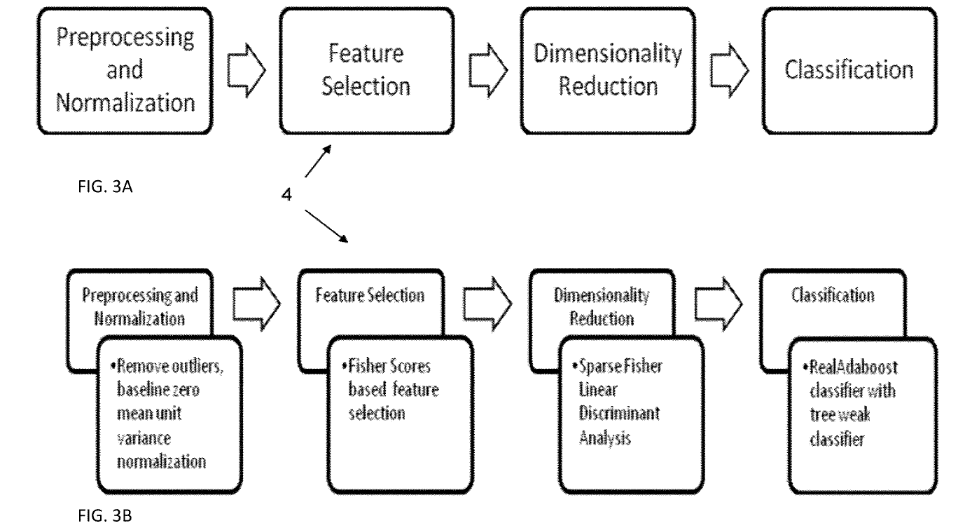

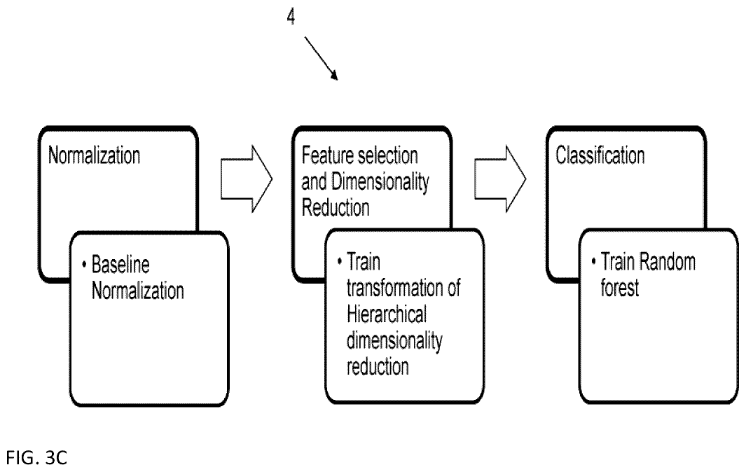

The embodiments shown in FIG. 1 are merely for conceptual clarity and not by way of limitation. The operation and elements of the system 200 for training a machine learning model 215 for use in a sensory monitoring device may be the same for a system for training a machine learning model for use in a pain monitoring device (PMD). Thus, the operation and elements of a pain monitoring device may be used as an exemplary embodiment of a sensory monitoring device, particularly in FIGS. 2 and 3A-3C hereinbelow.

Note that the operation of the at least one first SMD 285 of the at least one first SMD type as described herein below may be substantially the same as that of the at least one second SMD 285 of the at least one second SMD type. Thus, the training dataset to implement the training of the at least one trained second SMD MLM 255 may use for output dataset features, the at least one sensory metric output from the at least one first SMD 285 of the at least one first SMD type and for the input dataset features, the at the at least one sensor dataset from the at least one second SMD sensor for the at least one second SMD type. After training, the at least one trained second SMD MLM 255 may be trained to output the at least one second subject-specific sensory output metric in response to the sensor output from the at least one second sensors. This MLM may then be provided to the at least one second SMD 270 of the at least one second SMD type for use in subsequent medical procedures.

Referring to FIG. 2, the signal flow diagram 300 that may represent the signal processing implemented in the at least one first sensory monitoring device 285, 295 which may be configured to monitor pain. The signal flow diagram 300, for example, may include a physiological signal acquisition module 302 (e.g., acquire signal data from the at least one SMD sensor 283, 293) and a processing module 304 (e.g., the at least one SMD processor 284, 294) all configured for monitoring pain. The signal acquisition module 302 configured to receive a signal data from at least one sensor or transducers and may be used for acquiring and measuring physiological measurements from a subject able to experience pain. The processing module 304 may be include a plurality of sub-modules such as, for example, but not limited to signal processing module 2, feature extraction module 3, and/or machine learning module 4.

Note that modules and/or elements shown in FIG. 1 when configured for pain monitoring, may be subsequently related interchangeably to the embodiments shown in FIGS. 2 and 3A-3C.

In some embodiments, the signal flow diagram 300 may represent the signal processing implemented in the at least one second SMD 270 of the at least one second SMD type.

In some embodiments, the machine learning module 4 of FIG. 2 (e.g., the first SMD MLM 215, and/or the trained second SMD MLM 255, for example) may include at least one trained machine learning model, at least one trained algorithm, or both configured to output the at least one subject-specific sensory output metric indicative of a level of sensory event experienced by at least one subject during the subject-specific medical procedure. In other embodiments, the at least one subject-specific sensory output metric may be the at least one pain metric dataset, where the level of sensory event may be a level of pain experienced by the at least one subject during the subject-specific medical procedure.

In some embodiments, the signal processing module 2 may preprocess the acquired signal data, for example, that may include but not limited to normalization, filtering, noise reduction, SNR optimization, domain transformations, statistical analysis, spectral analysis, wavelet analysis, and the like.

In some embodiments, the feature extraction module 3 may extract features associated with the signal data (e.g., sensor signal data output from the at least one first SMD sensor 283, 293, for example) acquired by the physiological signal acquisition module 302.

In some embodiments, the feature extraction module 3 may further include an a priori data sub-module 6 for providing additional data beyond the acquired signals, for example, including but not limited to physician and/or caregiver data, medical history, genetic predispositions data, medical records, or the like a priori data.

In some embodiments, the feature extraction module 3 may perform signal processing techniques to extract from a physiological signal relevant and pertinent data. For example, an ECG measured signal that may be acquired in the physiological signal acquisition module 302 may be processed to provide a plurality of features, for example, including but not limited to HR, HRV, complex analysis, cardiac output data, or the like.

In some embodiments, the output module 5 may be provided to display or otherwise communicate the results provided by machine learning module 4.

In some embodiments, the at least one sensory metric output results may be displayed in a plurality of formats, for example, including but not limited to a printout, visual display cues, acoustic cues, or the like. In other embodiments, the results may be displayed in graded or class formats, or the like.

In some embodiments, the output module 5 may communicate the results to an external device for further processing, medical intervention, or the like, for example, results may be communicated to a drug administration device to automatically, semi-automatically, or manually control the drug delivery of a pain medication, or to indicate to and/or alert a caregiver to control, change, decrease, or increase the dosage or delivery of a pain-reducing drug.

In some embodiments, the output module 5 may control the drug administration device to administer a pain medication and/or any other suitable medication to the subject in the following scenarios:
Operating room—controlling the delivery of anesthetics and\or analgesics and\or hemodynamic drugs
Intensive care unit—controlling the delivery of anesthetics and\or analgesics and\or hemodynamic drugs
Post Anesthesia Care Unit—controlling pain medication administration and\or hemodynamic drugs
Patient Ward—provide longer term monitoring of patient status and control the delivery of pain medication and\or hemodynamic drugs
Pain clinic—alleviating chronic pain by monitoring the patient and controlling the dosage of pain medications

In some embodiments, each of a plurality of N pain monitoring devices, that may be represented by the First SMD 1 285 . . . the First SMD N 295 configured for pain monitoring, may include the machine learning module 4 to classify the feature vector set provided from the feature extraction module 3.

In some embodiments, the input features from the feature extraction module 3 based at least in part on at least one measured physiological signal may include, but are not limited to:
1. PPG Peak (P) and Trough (T) amplitude, mean amplitude, and standard deviation (STD) of amplitude
2. PPG Maximum Rate (MR) point
3. PPG Dicrotic Notch
4. PPG PP/PT/PN/NT/NM intervals, mean and STD (variability) of interval
5. PPG P-P Variability Frequency (PPG-HRV)
6. PPG P-P Variability Frequency LF/HF
7. PPG Pulse Area Under Curve (AUC)
8. Spectrum PPG Envelope
9. PPG Variability Wavelet Analysis
10. PPG-RSA (Respiratory Sinus Arrhythmia)
11. GSR Amplitude, Mean Amplitude, and STD of Amplitude
12. GSR Peak (P) Amplitude, Mean Amplitude, and STD of Amplitude
13. GSR PP Interval, Mean and STD (variability) of Interval
14. GSR Phasic EDA Amplitude, Mean Amplitude, and STD of Amplitude
15. GSR Spectrum: Spectrum of the GSR Signal
16. GSR Peak Amplitude
17. GSR Peak Frequency
18. GSR Wavelet Analysis

The following 9 possible input features may be derived directly from a ECG signal or estimated using alternative sensors, including but not limited to remote sensors, as described above:
19. ECG Q/R/S/T/P Amplitude, Mean and STD of Amplitude
20. ECG RR/PQ/PR/QT/RS/ST Interval, Mean and STD (variability) of Interval
21. ECG R-R Variability (ECG-HRV) VLF, LF, MF, and HF
22. ECG R-R Variability LF/HF
23. ECG-RSA (Respiratory Sinus Arrhythmia
24. ECG RRI Variability Wavelet Analysis
25. ECG Alpha: Slope of HRV power spectrum in a predefined time window.
26. ECG Beta: Slope of the log of HRV power spectrum in a predefined time window.
27. ECG-PPG PTT (Pulse Transition Time)
28. Temperature Amplitude, Mean Amplitude, and STD of Amplitude
29. Temperature Peak (P) Amplitude, Mean Amplitude, and STD of Amplitude
30. Temperature PP Interval, Mean and STD (variability) of Interval
31. Temperature Spectrum of the Temperature Signal
32. Temperature Peak Amplitude: The amplitude of the highest peak of the power spectrum in a predefined time window.
33. Temperature Peak Frequency: The frequency of the highest peak of the power spectrum in a predefined time window.
34. Temperature Upper Peak Amplitude, Mean Amplitude, and STD of Amplitude
35. Temperature Lower Peak Amplitude, Mean Amplitude, and STD of Amplitude
36. Respiratory Rate, Mean Rate, and STD Rate
37. Blood Pressure (BP) Peak (P) and Trough (T) Amplitude, Mean Amplitude, and
STD of Amplitude: The amplitude of the Peak (P) and the Trough (T) of the BP signal, mean amplitudes, and STD of amplitudes in a predefined time window. Peak denotes systolic blood pressure; Trough denotes diastolic blood pressure.
38. EEG Power of α, β, γ, δ, θ Frequency Bands: Power of the frequency bands of the power spectrum of EEG/fEMG signal in a predefined time segment. Frequency range of different bands [Hz]: delta, δ 0.5-4; theta, θ 4-8; alpha, α 8-14; beta, β 14-30; gamma γ, 30-70.
39. EEG Mean Frequency: The sum of the product of the power spectrum values in a predefined time segment and the frequencies, divided by the total power.
40. EEG Peak Frequency: The frequency of the highest peak of the power spectrum in a predefined time segment.
41. EEG Spectral Edge/Frequency
42. EEG Approximate Entropy
43. BSR-Burst Suppression Ratio
44. BcSEF: Burst compensated spectral edge frequency
45. WSMF: weighted spectral median frequency (WSMF)
46. CUP: Canonical univariate parameter
47. SpEn: Spectral Entropy
48. BcSpEn: Burst compensated Spectral Entropy
50. Histogram Parameters: Mean, Standard deviation, Kurtosis, Skewness of signal histogram in a predefined time segment.
51. AR Parameters
52. Normalized Slope Descriptors (Hjorth Parameters)
53. Barlow Parameters
54. Wackermann Parameters
55. Brain Rate
57. 80 Hz Frequency in EEG near the Eyes
58. EMG Spectrum Analysis: Power of the frequency bands of the power spectrum of EMG signal in a predefined time segment. Frequency range of different bands [Hz].
59. Mean Frequency: The sum of the product of the power spectrum values in a predefined time segment and the frequencies, divided by the total power.
60. Peak Frequency: The frequency of the highest peak of the power spectrum in a predefined time segment.
61. Total Power: The sum of the power spectrum within the epoch.
62. Spontaneous Lower Oesophageal Contractions (SLOC): Lower oesophageal contractility (LOC)
63. Average/Variability of End Tidal Carbon Dioxide
64. Average of End Tidal Sevoflurane
65. Average Value, Variability of Accelerometer X, Y, Z, Theta: Accelerometer X, Y, Z theta, movement analysis.
66. Facial expression images and\or video.
67. Pupilomety
68. EOG

In some embodiments, these input features based on at least one measured physiological signal may be used by the machine learning module 4 that is trained to output pain classification and/or pain metrics. The machine learning module 4 may analyze the input features to identify patterns and/or correlations that are indicative of different levels of pain. By processing these features, the machine learning module 4 may classify the pain into various categories and/or generate pain metrics that may provide a quantitative measure of the pain experienced by the subject.

The at least one output pain metric outputted by the machine learning module 4 may include, but are not limited to:
1. Pain Intensity Level: A numerical value representing the intensity of pain experienced by the subject, typically on a scale from 0 to 10 or 0 to 100.
2. Pain Classification: Categorization of pain into different classes such as no pain, mild pain, moderate pain, and severe pain.
3. Pain Duration: The duration of time the subject has been experiencing pain.
4. Pain Frequency: The frequency of pain episodes over a specified period.
5. Pain Variability: The variability in pain intensity over time.
6. Pain Onset: The time at which the pain began.
7. Pain Peak: The maximum intensity of pain experienced during a pain episode.
8. Pain Relief: The reduction in pain intensity following an intervention or treatment.
9. Pain Trend: The trend in pain intensity over time, indicating whether the pain is increasing, decreasing, or stable.
10. Pain Correlation: Correlation of pain intensity with specific physiological or behavioral features.

In some embodiments, the output pain metrics may also include a nociception factor, or a nociception metric.

In some embodiment, any of the input features from the feature extraction module 3 (e.g, second SMD feature extraction module 212) in the input feature list above and any of the output pain metrics in the list above for a particular subject generated by the at least one first SMD may be used by the MLM training module 217 to generate the at least one training dataset

for the plurality of subjects that include the first SMD sensory output metric dataset 232 and the sensor dataset 234 for the at least one second SMD type as shown in FIG. 1.

In some embodiments, a Great Plurality of Features (GPF) may be a comprehensive collection of physiological features utilized for detecting, classifying, and identifying pain levels in a subject. The GPF may be a wide array of features extracted from various physiological signals (e.g., as in the input feature list hereinabove), that may include but are not limited to Photoplethysmograph (PPG), Galvanic Skin Response (GSR), skin temperature, Electrocardiogram (ECG), respiration, Electromyography (EMG), and Electroencephalography/Frontalis Electromyography (EEG/FEMG). The GPF may be used to capture a multifaceted nature of physiological responses to pain, incorporating both temporal and spectral characteristics of the signals. By leveraging a diverse set of features, the GPF may facilitate a robust pain classification across various states of consciousness, including sedated, partially sedate, and awake conditions. This holistic approach may ensure that the system may effectively monitor and detect pain, providing valuable insights for clinical decision-making and patient care.

In some embodiments, feature extraction module 3 may form the GPF vector for further analysis, for example, using the above input features used to generate the output pain metrics listed hereinabove.

In some embodiments, generating a pain metric from the GPF (e.g., from the pain metric list hereinabove) may require a comprehensive set of input features extracted from at least one or more physiological signals. The exact number of features may vary depending on the specific implementation and the complexity of the pain classification model.

With regard to FIG. 1, for the processor 210 to construct at least one training dataset using datasets collected from the plurality of subjects, the machine learning module 217 may map the input features (e.g., input features vectors) to the output subject-specific sensory metrics for each of the plurality of subjects such as for example, subject-specific pain metrics. The generation of the training dataset, and subsequent training using the MLM Training module 217 to generate the trained second SMD MLM 255 may involve one or more of the following steps:
1. Data Collection: Collect a comprehensive dataset from a diverse population of subjects representing the target population, including raw signal data and corresponding pain metrics.
2. Data Preprocessing: Clean the dataset to remove noisy, erroneous and\or irrelevant information. Optionally, normalize the data to ensure consistency across different measurements and subjects. More importantly, in this step we need to handle missing or low quality data.
3. Feature Extraction: Extract relevant features from the raw data using signal processing techniques. This step is optional in some ML techniques.
4. Splitting the Dataset: Divide the dataset into training, validation, and test sets to evaluate the model's performance.
5. Model Training: Train the machine learning model using the training dataset, optimizing the model's parameters to accurately map the input features to the output pain metrics.
6. Model Testing: Test the model's performance on the test dataset to ensure it generalizes well to new, unseen data.

In some embodiments, one model training and testing example may include “Leave One Subject Out” (LOSO) in which the training may be done iteratively over all the data except for one subject that the trained model is tested against. This may be done iteratively leaving one subject out each time until all subjects are tested for.

In some embodiments, FIGS. 3A-C may provide further optional depictions of machine learning module 4 of the processing module 304 in at least one first pain-monitoring device (e.g., that may be represented by the at least one first SMD-1 285 . . . the at least one first SMD-N of FIG. 1 for pain monitoring) and/or the at least one second SMD 270 wherein the feature vector set provided from module 3 may be processed, such as for example, with a plurality of machine learning sub-modules. These submodules may include for example, a preprocessing and normalization sub-module, a feature selection and dimensionality reduction sub-module, and/or a classification sub-module. Machine-learning sub-modules may be implemented by any suitable machine learning techniques and methods. Optionally, these sub-modules of the machine learning module (e.g., preprocessing and normalization, feature selection, and dimensionality reduction) may be reorganized and provided in all possible combinations.

FIG. 3B may provide a depiction of a non-limiting example of an optional embodiment of FIG. 3A, also described in some embodiments below, wherein the machine learning module 4 of the processing module 304 of FIG. 2 may be provided by a collection of machine learning sub-modules. For example, preprocessing may be provided by a plurality of actions including outlier removal and signal normalization utilizing baseline zero mean unit variance; feature selection may be provided through the use of a Fisher score; dimensionality reduction may be provided by Sparse Fisher Linear Discriminant Analysis (SFLDA) while classification may be provided by a RealAdaboost classifier.

In some embodiments, the machine learning module 4 of FIG. 2 may include at least one preprocessing sub-module, at least one feature selection and dimensionality reduction sub-module, and/or at least one classification sub-module in any combination thereof.

FIG. 3C may provide a depiction of a non-limiting example of an optional embodiment of FIG. 3A, also described in some embodiments below, wherein the machine learning module 4 of the processing module 304 of FIG. 2 may be provided by a collection of machine learning sub-modules. For example, preprocessing may be provided by a plurality of actions including outlier removal and signal normalization utilizing baseline zero mean unit variance; feature selection and dimensionality reduction may be provided by Hierarchical dimensionality reduction while classification may be provided by a Random Forest classifier.

In some embodiments, the machine learning module 4 may provide for feature selection and dimensionality reduction wherein the GPF vector may undergo feature selection and dimensionality reduction to provide a second vector therein providing a representation of the GPF in a smaller dimension.

In some embodiments, feature selection and dimensionality reduction techniques, for example, may include but are not limited to Multi-Dimensional Scaling (MDS), Principal Component Analysis (PCA), Sparse PCA (SPCA), Fisher Linear Discriminant Analysis (FLDA), Sparse FLDA (SFLDA), Kernel PCA (KPCA), ISOMAP, Locally Linear Embedding (LLE), Laplacian Eigenmaps, Diffusion Maps, Hessian Eigenmaps, Independent Component Analysis (ICA), Factor analysis (FA), Hierarchical Dimensionality Reduction (HDR), Sure Independence Screening (SIS), Fisher score ranks, t-test rank, Mann-Whitney U-test taken alone or in any combination thereof, or as known and accepted in the art.

In some embodiments, classification may be performed on at least one of the GPF vector or a second vector that may include a dimensionally reduced version of GPF vector.

In some embodiments, machine learning module 4 may provide for pain classification into at least two or more classes, for example, including but not limited to pain and non-pain groups.

In some embodiments, a plurality of optional classifiers may be used, for example, including but not limited to Recurrent Neural Network (RNN), Long Short Term Memory (LSTM), Convolutional Neural Networks (CNN), Gated Recurrent Unit (GRU), Generative Adversarial Network (GAN), Nearest Shrunken Centroids (NSC), Classification and Regression Trees (CART), ID3, C4.5, Multivariate Additive regression splines (MARS), Multiple additive regression trees (MART), Nearest Centroid (NC), Shrunken Centroid Regularized Linear Discriminate and Analysis (SCRLDA), Random Forest, Boosting, Bagging Classifier, AdaBoost, RealAdaBoost, LPBoost, TotalBoost, BrownBoost, MadaBoost, LogitBoost, GentleBoost, RobustBoost, Support Vector Machine (SVM), kernelized SVM, Linear classifier, Quadratic Discriminant Analysis (QDA) classifier, Naïve Bayes Classifier and Generalized Likelihood Ratio Test (GLRT) classifier with plug-in parametric or non-parametric class conditional density estimation, k-nearest neighbor, Radial Base Function (RBF) classifier, Multilayer Perceptron classifier, Bayesian Network (BN) classifier, multi-class classifier adapted from binary classifier with one-vs-one majority voting, one-vs-rest, Error Correcting Output Codes, hierarchical multi-class classification, Committee of classifiers or the like as are known and accepted in the art or any combination thereof.

In some embodiments, classification may be implemented with a graded scoring that may score the classified level of pain.

In some embodiments, data collection may involve collecting a comprehensive dataset from a diverse population of subjects, including physiological signals (e.g., PPG, EEG, ECG, EOG, EMG, pupillometry, ..., heart rate, blood pressure, skin conductance) and\or behavioral expressions (e.g., facial expressions, body movements), and simultaneously collecting the output of a commercially available pain/nociception monitor attached to the same subjects, that may serve as ground truth for training an AI model (e.g., using the output 286, 296 from the at least one first SMD 285, 295)

In some embodiments, data preprocessing, that may be implemented for example by data filtering module 213, may involve cleaning the dataset to remove noisy and irrelevant information. Optionally, normalizing the data to ensure consistency across different measurements and subjects.

In some embodiments, a model selection step may be performed. Model selection may involve evaluating various Machine Learning algorithms (e.g., random forest, neural networks, support vector machines) and/or Deep Learning techniques (e.g., CNN, RNN, LSTM) to determine the most effective model for pain prediction and/or for general sensory monitoring.

In some embodiments, model training may involve training the selected AI model using the preprocessed dataset.

In some embodiments, model implementation may involve integrating the trained AI model into sensory\pain monitoring devices and/or testing the model in clinical settings to assess its real-world applicability and accuracy.

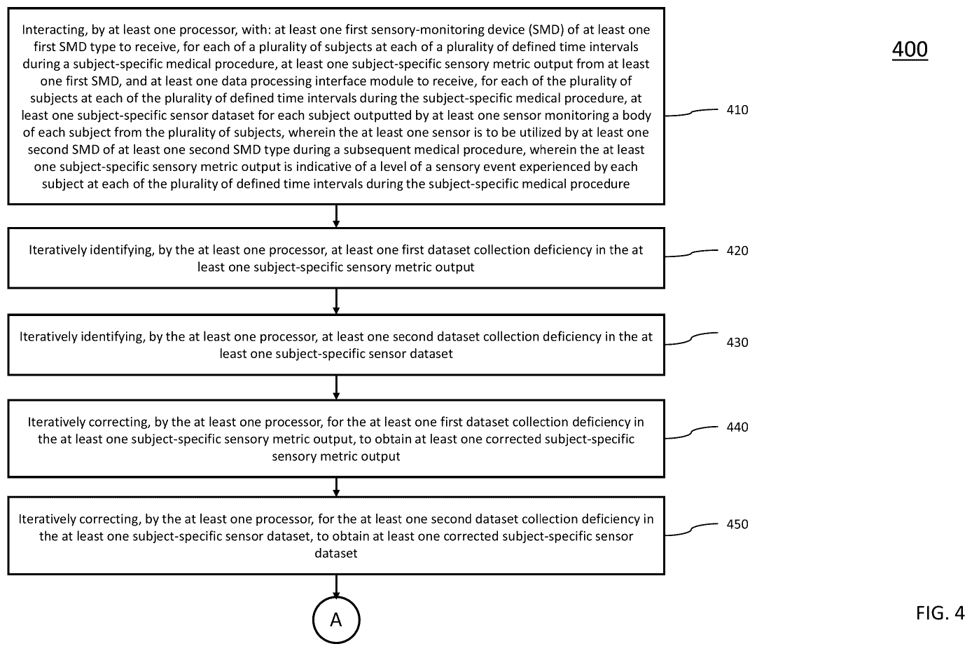

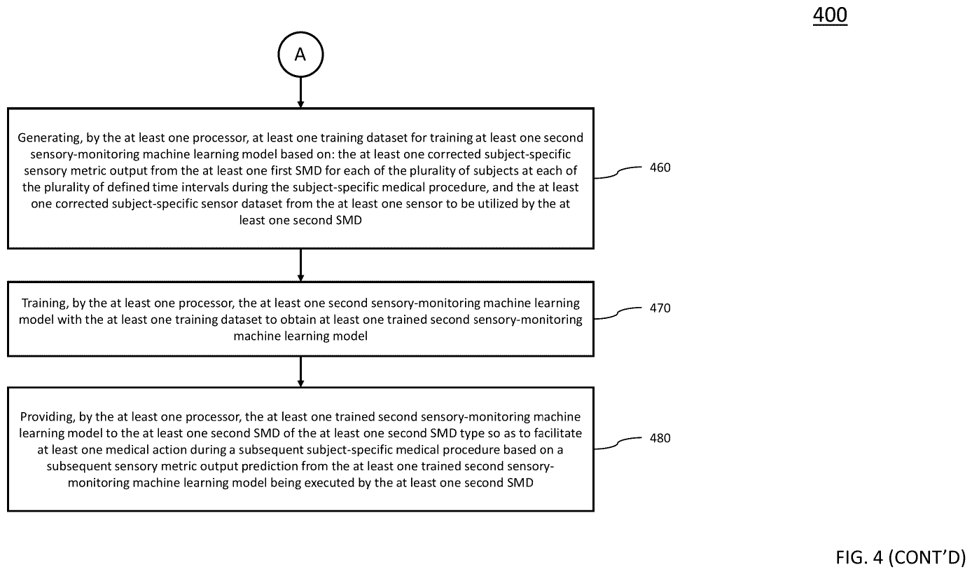

FIG. 4 is a flowchart of a method 400 for training a machine learning model for use in a sensory monitoring device (e.g., the at least one second SMD 270) according to the present disclosure. The method may be performed by the processor 210, for example.

In some embodiments, the method 400 may include interacting 410, by at least one processor, with: at least one first sensory-monitoring device (SMD) of at least one first SMD type to receive, for each of a plurality of subjects at each of a plurality of defined time intervals during a subject-specific medical procedure, at least one subject-specific sensory metric output from at least one first SMD, and at least one data processing interface module to receive, for each of the plurality of subjects at each of the plurality of defined time intervals during the subject-specific medical procedure, at least one subject-specific sensor dataset for each subject outputted by at least one sensor monitoring a body of each subject from the plurality of subjects, wherein the at least one sensor is to be utilized by at least one second SMD of at least one second SMD type during a subsequent medical procedure, wherein the at least one subject-specific sensory metric output is indicative of a level of a sensory event experienced by each subject at each of the plurality of defined time intervals during the subject-specific medical procedure.

In some embodiments, the method 400 may include iteratively identifying 420, by the at least one processor, at least one first dataset collection deficiency in the at least one subject-specific sensory metric output.

In some embodiments, the method 400 may include iteratively identifying 430, by the at least one processor, at least one second dataset collection deficiency in the at least one subject-specific sensor dataset.

In some embodiments, the method 400 may include iteratively correcting 440, by the at least one processor, for the at least one first dataset collection deficiency in the at least one subject-specific sensory metric output, to obtain at least one corrected subject-specific sensory metric output.

In some embodiments, the method 400 may include iteratively correcting 450, by the at least one processor, for the at least one second dataset collection deficiency in the at least one subject-specific sensor dataset, to obtain at least one corrected subject-specific sensor dataset.

In some embodiments, the method 400 may include generating 460, by the at least one processor, at least one training dataset for training at least one second sensory-monitoring machine learning model based on: the at least one corrected subject-specific sensory metric output from the at least one first SMD for each of the plurality of subjects at each of the plurality of defined time intervals during the subject-specific medical procedure, and the at least one corrected subject-specific sensor dataset from the at least one sensor to be utilized by the at least one second SMD.

In some embodiments, the method 400 may include training 470, by the at least one processor, the at least one second sensory-monitoring machine learning model with the at least one training dataset to obtain at least one trained second sensory-monitoring machine learning model.

In some embodiments, the method 400 may include providing 480, by the at least one processor, the at least one trained second sensory-monitoring machine learning model to the at least one second SMD of the at least one second SMD type so as to facilitate or cause at least one medical action during a subsequent subject-specific medical procedure based on a subsequent sensory metric output prediction from the at least one trained second sensory-monitoring machine learning model being executed in real time by the at least one second SMD.

In some embodiments, the at least one first sensory-monitoring device (SMD) may be at least one first pain monitoring device (PMD).

In some embodiments, the at least one second sensory-monitoring device (SMD) may be at least one second pain monitoring device (PMD).

In some embodiments, the at least one medical action based on the sensory metric output prediction from the at least one trained second sensory-monitoring machine learning model may include results being communicated to a drug administration device to automatically, semi-automatically, or manually control the drug delivery of a pain medication. The at least one medical action may include indicating to and/or alerting a caregiver to control, change, decrease, or increase the dosage or delivery of a pain-reducing drug. The at least one medical action may include an operating room scenario—controlling the delivery of anesthetics and/or analgesics and/or hemodynamic drugs. The at least one medical action may include an intensive care unit scenario—controlling the delivery of anesthetics and/or analgesics and/or hemodynamic drugs. The at least one medical action may include in a Post Anesthesia Care Unit scenario - controlling pain medication administration and/or hemodynamic drugs. The at least one medical action may include in a Patient Ward scenario—providing longer term monitoring of patient status and controlling the delivery of pain medication and/or hemodynamic drugs. The at least one medical action may include in a Pain clinic scenario—alleviating chronic pain by monitoring the patient and controlling the dosage of pain medications.

At least some embodiments disclosed address a technical problem where training new device models may require having a reliable “ground truth” for training large scale new models, such as in machine learning and artificial intelligence. The technical solution to this technical problem may include using at least one first sensory-monitoring device to generate subject-specific sensory metric outputs and time-aligned sensor datasets that may serve as training data for at least one second sensory-monitoring device. The at least one processor may train at least one second sensory-monitoring machine learning model using the first-device outputs as reference labels and may provide the at least one trained second sensory-monitoring machine learning model to the at least one second SMD of the at least one second SMD type so as to cause at least one medical action during a subsequent subject-specific medical procedure responsive to a subsequent sensory metric output prediction generated in real time by execution of the at least one trained second sensory-monitoring machine learning model by the at least one second SMD.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Matlab, Perl, QT, etc.).

In some embodiments, one or more of exemplary inventive computer-based systems/platforms, exemplary inventive computer-based devices, and/or exemplary inventive computer-based components of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of exemplary inventive computer-based systems/platforms, exemplary inventive computer-based devices, and/or exemplary inventive computer-based components of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a social media post, a map, an entire application (e.g., a calculator), etc. In some embodiments, as detailed herein, one or more of exemplary inventive computer-based systems/platforms, exemplary inventive computer-based devices, and/or exemplary inventive computer-based components of the present disclosure may be implemented across one or more of various computer platforms such as, but not limited to: (1) FreeBSD, NetBSD, OpenBSD; (2) Linux; (3) Microsoft Windows; (4) OS X (MacOS); (5) MacOS 11; (6) Solaris; (7) Android; (8) iOS; (9) Embedded Linux; (10) Tizen; (11) WebOS; (12) IBM i; (13) IBM AIX; (14) Binary Runtime Environment for Wireless (BREW); (15) Cocoa (API); (16) Cocoa Touch; (17) Java Platforms; (18) JavaFX; (19) JavaFX Mobile; (20) Microsoft DirectX; (21) .NET Framework; (22) Silverlight; (23) Open Web Platform; (24) Oracle Database; (25) Qt; (26) Eclipse Rich Client Platform; (27) SAP NetWeaver; (28) Smartface; and/or (29) Windows Runtime.

In some embodiments, exemplary inventive computer-based systems/platforms, exemplary inventive computer-based devices, and/or exemplary inventive computer-based components of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software.

For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, exemplary inventive computer-based systems/platforms, exemplary inventive computer-based devices, and/or exemplary inventive computer-based components of the present disclosure may be configured to handle numerous concurrent subjects that may be, but is not limited to, at least 100 (e.g., but not limited to, 100-999), at least 1,000 (e.g., but not limited to, 1,000-9,999) , at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, exemplary inventive computer-based systems/platforms, exemplary inventive computer-based devices, and/or exemplary inventive computer-based components of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display (may be a holographic display. In various implementations, the display may be a transparent surface that may receive a visual projection. Such projections may convey various forms of information, images, and/or objects. For example, such projections may be a visual overlay for a mobile augmented reality (MAR) application.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Pager, Smartphone, or any other reasonable mobile electronic device.

As used herein, the terms “proximity detection,” “locating,” “location data,” “location information,” and “location tracking” refer to any form of location tracking technology or locating method that can be used to provide a location of, for example, a particular computing device/system/platform of the present disclosure and/or any associated computing devices” (e.g., for tracking movements of the leg 25), based at least in part on one or more of the following techniques/devices, without limitation: accelerometer(s), gyroscope(s), Global Positioning Systems (GPS); GPS accessed using Bluetooth™; GPS accessed using any reasonable form of wireless and/or non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed; this is in no way meant to be a limitation.

As used herein, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the exemplary inventive computer-based systems/platforms, the exemplary inventive computer-based devices, and/or the exemplary inventive computer-based components of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

The aforementioned examples are, of course, illustrative and not restrictive.

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

In some embodiments, the exemplary inventive computer-based systems/platforms, the exemplary inventive computer-based devices, and/or the exemplary inventive computer-based components of the present disclosure may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows:
i) Define Neural Network architecture/model,
ii) Transfer the input data to the exemplary neural network model,
iii) Train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated.

In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, a method may include: interacting, by at least one processor, with: at least one first sensory-monitoring device (SMD) of at least one first SMD type to receive, for each of a plurality of subjects at each of a plurality of defined time intervals during a subject-specific medical procedure, at least one subject-specific sensory metric output from at least one first SMD, and at least one data processing interface module to receive, for each of the plurality of subjects at each of the plurality of defined time intervals during the subject-specific medical procedure, at least one subject-specific sensor dataset for each subject outputted by at least one sensor monitoring a body of each subject from the plurality of subjects; where the at least one sensor is to be utilized by at least one second SMD of at least one second SMD type during a subsequent medical procedure; where the at least one subject-specific sensory metric output may be indicative of a level of a sensory event experienced by each subject at each of the plurality of defined time intervals during the subject-specific medical procedure; iteratively identifying, by the at least one processor, at least one first dataset collection deficiency in the at least one subject-specific sensory metric output; iteratively identifying, by the at least one processor, at least one second dataset collection deficiency in the at least one subject-specific sensor dataset; iteratively correcting, by the at least one processor, for the at least one first dataset collection deficiency in the at least one subject-specific sensory metric output, to obtain at least one corrected subject-specific sensory metric output; iteratively correcting, by the at least one processor, for the at least one second dataset collection deficiency in the at least one subject-specific sensor dataset, to obtain at least one corrected subject-specific sensor dataset; generating, by the at least one processor, at least one training dataset for training at least one second sensory-monitoring machine learning model based on: the at least one corrected subject-specific sensory metric output from the at least one first SMD for each of the plurality of subjects at each of the plurality of defined time intervals during the subject-specific medical procedure, and the at least one corrected subject-specific sensor dataset from the at least one sensor to be utilized by the at least one second SMD; training, by the at least one processor, the at least one second sensory-monitoring machine learning model with the at least one training dataset to obtain at least one trained second sensory-monitoring machine learning model; and providing, by the at least one processor, the at least one trained second sensory-monitoring machine learning model to the at least one second SMD of the at least one second SMD type so as to cause at least one medical action during a subsequent subject-specific medical procedure responsive to a subsequent sensory metric output prediction generated in real time by execution of the at least one trained second sensory-monitoring machine learning model by the at least one second SMD.

In some embodiments, the at least one second SMD may be the at least one first SMD. the at least one second SMD type may be the at least one first SMD type, or the at least one second SMD type may be different than the at least one first SMD type.

In some embodiments, the at least one second SMD type may be the at least one first SMD type. The at least one second SMD and the at least one first SMD, or the at least one second SMD is different than the at least one first SMD.

In some embodiments, the at least one first dataset collection deficiency may include deficiencies during the subject-specific medical procedure caused by a patient movement, an interference from other medical equipment, a sensor malfunction, a sensor misplacement on the body, a usage of electric cautery, a usage of diathermy, a usage of vasoactive drugs, manipulations that may affect physiological markers, or any combination thereof.

In some embodiments, the at least one second dataset collection deficiency may include deficiencies during the subject-specific medical procedure caused by a patient movement, an interference from other medical equipment, a sensor malfunction, a sensor misplacement on the body, a usage of electric cautery, a usage of diathermy, a usage of vasoactive drugs, manipulations that may affect physiological markers, or any combination thereof.

In some embodiments, iteratively correcting the at least one first dataset collection deficiency may include iteratively correcting low quality input data, erroneous data, missing input data, or any combination thereof, in the at least one subject-specific sensory metric output.

In some embodiments, iteratively correcting the at least one second dataset collection deficiency may include iteratively correcting low quality input data, erroneous data, missing input data, or any combination thereof, in the at least one subject-specific sensor dataset.

In some embodiments, the at least one first SMD may include: at least one first SMD sensor to monitor the body of each subject; and at least one first SMD processor that may be configured to execute at least one trained first sensory-monitoring machine learning model; where the at least one trained first sensory-monitoring machine learning model may be trained to output the at least one subject-specific sensory metric output for an input of at least one subject-specific first SMD sensor data from the at least one first SMD sensor; and where the interacting with the at least one first SMD to receive the at least one subject-specific sensory metric output may include inputting the at least one subject-specific first SMD sensor data to the at least one first SMD to receive the at least one subject-specific sensory metric output.

In some embodiments, the at least one first SMD may include at least one first pain monitoring device; where the at least one second SMD may be at least one second pain monitoring device; and where the at least one subject-specific sensory metric output may be a pain metric indicative of a level of pain experienced by each subject during the subject-specific medical procedure.

In some embodiments, the pain metric may be a nociception metric.

In some embodiments, a system may include at least one processor; and at least one non-transitory memory. The at least one processor may be configured to execute computer code that causes the at least one processor to: interact with: at least one first sensory-monitoring device (SMD) of at least one first SMD type to receive, for each of a plurality of subjects at each of a plurality of defined time intervals during a subject-specific medical procedure, at least one subject-specific sensory metric output from at least one first SMD, and at least one data processing interface module to receive, for each of the plurality of subjects at each of the plurality of defined time intervals during the subject-specific medical procedure, at least one subject-specific sensor dataset for each subject outputted by at least one sensor monitoring a body of each subject from the plurality of subjects. The at least one sensor is to be utilized by at least one second SMD of at least one second SMD type during a subsequent medical procedure. The at least one subject-specific sensory metric output may be indicative of a level of a sensory event experienced by each subject at each of the plurality of defined time intervals during the subject-specific medical procedure. The at least one processor may be configured to iteratively identify at least one first dataset collection deficiency in the at least one subject-specific sensory metric output, iteratively identify at least one second dataset collection deficiency in the at least one subject-specific sensor dataset, iteratively correct for the at least one first dataset collection deficiency in the at least one subject-specific sensory metric output, to obtain at least one corrected subject-specific sensory metric output, iteratively correct for the at least one second dataset collection deficiency in the at least one subject-specific sensor dataset, to obtain at least one corrected subject-specific sensor dataset, generate at least one training dataset for training at least one second sensory-monitoring machine learning model based on: the at least one corrected subject-specific sensory metric output from the at least one first SMD for each of the plurality of subjects at each of the plurality of defined time intervals during the subject-specific medical procedure, and the at least one corrected subject-specific sensor dataset from the at least one sensor to be utilized by the at least one second SMD. The at least one processor may be configured to train the at least one second sensory-monitoring machine learning model with the at least one training dataset to obtain at least one trained second sensory-monitoring machine learning model and provide the at least one trained second sensory-monitoring machine learning model to the at least one second SMD of the at least one second SMD type so as to cause at least one medical action during a subsequent subject-specific medical procedure responsive to a subsequent sensory metric output prediction generated in real time by execution of the at least one trained second sensory-monitoring machine learning model by the at least one second SMD.

In some embodiments, the at least one second SMD may be the at least one first SMD. the at least one second SMD type may be the at least one first SMD type, or the at least one second SMD type may be different than the at least one first SMD type.

In some embodiments, the at least one second SMD type may be the at least one first SMD type. The at least one second SMD and the at least one first SMD, or the at least one second SMD is different than the at least one first SMD.

In some embodiments, the at least one second SMD of the at least one second SMD type may be different from the at least one first SMD of the at least one first SMD type.

In some embodiments, the at least one first dataset collection deficiency may include deficiencies during the subject-specific medical procedure caused by a patient movement, an interference from other medical equipment, a sensor malfunction, a sensor misplacement on the body, a usage of electric cautery, a usage of diathermy, a usage of vasoactive drugs, manipulations that may affect physiological markers, or any combination thereof.

In some embodiments, the at least one second dataset collection deficiency may include deficiencies during the subject-specific medical procedure caused by a patient movement, an interference from other medical equipment, a sensor malfunction, a sensor misplacement on the body, a usage of electric cautery, a usage of diathermy, a usage of vasoactive drugs, manipulations that may affect physiological markers, or any combination thereof.

In some embodiments, the at least one processor may be configured to iteratively correct the at least one first dataset collection deficiency by iteratively correcting low quality input data, erroneous data, missing input data, or any combination thereof, in the at least one subject-specific sensory metric output.

In some embodiments, the at least one processor may be configured to iteratively correct the at least one second dataset collection deficiency by iteratively correcting low quality input data, erroneous data, missing input data, or any combination thereof, in the at least one subject-specific sensor dataset.

In some embodiments, the at least one first SMD may include: at least one first SMD sensor to monitor the body of each subject; and at least one first SMD processor that is configured to execute at least one trained first sensory-monitoring machine learning model. The at least one trained first sensory-monitoring machine learning model may be trained to output the at least one subject-specific sensory metric output for an input of at least one subject-specific first SMD sensor data from the at least one first SMD sensor. The at least one processor may be configured to interact with the at least one first SMD to receive the at least one subject-specific sensory metric output by inputting the at least one subject-specific first SMD sensor data to the at least one first SMD to receive the at least one subject-specific sensory metric output.

In some embodiments, the at least one first SMD may be at least one first pain monitoring device. The at least one second SMD may be at least one second pain monitoring device. The at least one subject-specific sensory metric output may be a pain metric indicative of a level of pain experienced by each subject during the subject-specific medical procedure.

In some embodiments, the pain metric may be a nociception metric.

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the inventive systems/platforms, and the inventive devices described herein can be utilized in any combination with each other. Further still, the various steps may be carried out in any desired order (and any desired steps may be added and/or any desired steps may be eliminated).

1. A method, comprising:
interacting, by at least one processor, with:
at least one first sensory-monitoring device (SMD) of at least one first SMD type to receive, for each of a plurality of subjects at each of a plurality of defined time intervals during a subject-specific medical procedure, at least one subject-specific sensory metric output from at least one first SMD, and
at least one data processing interface module to receive, for each of the plurality of subjects at each of the plurality of defined time intervals during the subject-specific medical procedure, at least one subject-specific sensor dataset for each subject outputted by at least one sensor monitoring a body of each subject from the plurality of subjects;
wherein the at least one sensor is to be utilized by at least one second SMD of at least one second SMD type during a subsequent medical procedure;
wherein the at least one subject-specific sensory metric output is indicative of a level of a sensory event experienced by each subject at each of the plurality of defined time intervals during the subject-specific medical procedure;
iteratively identifying, by the at least one processor, at least one first dataset collection deficiency in the at least one subject-specific sensory metric output;
iteratively identifying, by the at least one processor, at least one second dataset collection deficiency in the at least one subject-specific sensor dataset;
iteratively correcting, by the at least one processor, for the at least one first dataset collection deficiency in the at least one subject-specific sensory metric output, to obtain at least one corrected subject-specific sensory metric output;
iteratively correcting, by the at least one processor, for the at least one second dataset collection deficiency in the at least one subject-specific sensor dataset, to obtain at least one corrected subject-specific sensor dataset;
generating, by the at least one processor, at least one training dataset for training at least one second sensory-monitoring machine learning model based on:
the at least one corrected subject-specific sensory metric output from the at least one first SMD for each of the plurality of subjects at each of the plurality of defined time intervals during the subject-specific medical procedure, and
the at least one corrected subject-specific sensor dataset from the at least one sensor to be utilized by the at least one second SMD;
training, by the at least one processor, the at least one second sensory-monitoring machine learning model with the at least one training dataset to obtain at least one trained second sensory-monitoring machine learning model; and
providing, by the at least one processor, the at least one trained second sensory-monitoring machine learning model to the at least one second SMD of the at least one second SMD type so as to cause at least one medical action during a subsequent subject-specific medical procedure responsive to a subsequent sensory metric output prediction generated in real time by execution of the at least one trained second sensory-monitoring machine learning model by the at least one second SMD.

2. The method of claim 1, wherein the at least one second SMD is the at least one first SMD; and wherein the at least one second SMD type is the at least one first SMD type, or the at least one second SMD type is different than the at least one first SMD type.

3. The method of claim 1, wherein the at least one second SMD type is the at least one first SMD type; and wherein the at least one second SMD is the at least one first SMD, or the at least one second SMD is different than the at least one first SMD.

4. The method of claim 1, wherein the at least one first dataset collection deficiency comprises deficiencies during the subject-specific medical procedure caused by a patient movement, an interference from other medical equipment, a sensor malfunction, a sensor misplacement on the body, a usage of electric cautery, a usage of diathermy, a usage of vasoactive drugs, manipulations that may affect physiological markers, or any combination thereof.

5. The method of claim 1, wherein the at least one second dataset collection deficiency comprises deficiencies during the subject-specific medical procedure caused by a patient movement, an interference from other medical equipment, a sensor malfunction, a sensor misplacement on the body, a usage of electric cautery, a usage of diathermy, a usage of vasoactive drugs, manipulations that may affect physiological markers, or any combination thereof.

6. The method of claim 1, wherein iteratively correcting the at least one first dataset collection deficiency comprises iteratively correcting low quality input data, erroneous data, missing input data, or any combination thereof, in the at least one subject-specific sensory metric output.

7. The method of claim 1, wherein iteratively correcting the at least one second dataset collection deficiency comprises iteratively correcting low quality input data, erroneous data, missing input data, or any combination thereof, in the at least one subject-specific sensor dataset.

8. The method according to claim 1, wherein the at least one first SMD comprises:
at least one first SMD sensor to monitor the body of each subject; and
at least one first SMD processor that is configured to execute at least one trained first sensory-monitoring machine learning model;
wherein the at least one trained first sensory-monitoring machine learning model is trained to output the at least one subject-specific sensory metric output for an input of at least one subject-specific first SMD sensor data from the at least one first SMD sensor; and
wherein the interacting with the at least one first SMD to receive the at least one subject-specific sensory metric output comprises inputting the at least one subject-specific first SMD sensor data to the at least one first SMD to receive the at least one subject-specific sensory metric output.

9. The method of claim 1, wherein the at least one first SMD is at least one first pain monitoring device;
wherein the at least one second SMD is at least one second pain monitoring device; and
wherein the at least one subject-specific sensory metric output is a pain metric indicative of a level of pain experienced by each subject during the subject-specific medical procedure.

10. The method of claim 9, wherein the pain metric is a nociception metric.

11. A system, comprising:
at least one processor; and
at least one non-transitory memory;
wherein the at least one processor is configured to execute computer code that causes the at least one processor to:
interact with:
at least one first sensory-monitoring device (SMD) of at least one first SMD type to receive, for each of a plurality of subjects at each of a plurality of defined time intervals during a subject-specific medical procedure, at least one subject-specific sensory metric output from at least one first SMD, and
at least one data processing interface module to receive, for each of the plurality of subjects at each of the plurality of defined time intervals during the subject-specific medical procedure, at least one subject-specific sensor dataset for each subject outputted by at least one sensor monitoring a body of each subject from the plurality of subjects;
wherein the at least one sensor is to be utilized by at least one second SMD of at least one second SMD type during a subsequent medical procedure;
wherein the at least one subject-specific sensory metric output is indicative of a level of a sensory event experienced by each subject at each of the plurality of defined time intervals during the subject-specific medical procedure;
iteratively identify at least one first dataset collection deficiency in the at least one subject-specific sensory metric output;
iteratively identify at least one second dataset collection deficiency in the at least one subject-specific sensor dataset;
iteratively correct for the at least one first dataset collection deficiency in the at least one subject-specific sensory metric output, to obtain at least one corrected subject-specific sensory metric output;
iteratively correct for the at least one second dataset collection deficiency in the at least one subject-specific sensor dataset, to obtain at least one corrected subject-specific sensor dataset ;
generate at least one training dataset for training at least one second sensory-monitoring machine learning model based on:
the at least one corrected subject-specific sensory metric output from the at least one first SMD for each of the plurality of subjects at each of the plurality of defined time intervals during the subject-specific medical procedure, and
the at least one corrected subject-specific sensor dataset from the at least one sensor to be utilized by the at least one second SMD;
train the at least one second sensory-monitoring machine learning model with the at least one training dataset to obtain at least one trained second sensory-monitoring machine learning model; and
provide the at least one trained second sensory-monitoring machine learning model to the at least one second SMD of the at least one second SMD type so as to cause at least one medical action during a subsequent subject-specific medical procedure responsive to a subsequent sensory metric output prediction generated in real time by execution of the at least one trained second sensory-monitoring machine learning model by the at least one second SMD.

12. The system of claim 11, wherein the at least one second SMD is the at least one first SMD; and wherein the at least one second SMD type is the at least one first SMD type, or the at least one second SMD type is different than the at least one first SMD type.

13. The system of claim 11, wherein the at least one second SMD type is the at least one first SMD type; and wherein the at least one second SMD is the at least one first SMD, or the at least one second SMD is different than the at least one first SMD.

14. The system of claim 11, wherein the at least one first dataset collection deficiency comprises deficiencies during the subject-specific medical procedure caused by a patient movement, an interference from other medical equipment, a sensor malfunction, a sensor misplacement on the body, a usage of electric cautery, a usage of diathermy, a usage of vasoactive drugs, manipulations that may affect physiological markers, or any combination thereof.

15. The system of claim 11, wherein the at least one second dataset collection deficiency comprises deficiencies during the subject-specific medical procedure caused by a patient movement, an interference from other medical equipment, a sensor malfunction, a sensor misplacement on the body, a usage of electric cautery, a usage of diathermy, a usage of vasoactive drugs, manipulations that may affect physiological markers, or any combination thereof.

16. The system of claim 11, wherein the at least one processor is configured to iteratively correct the at least one first dataset collection deficiency by iteratively correcting low quality input data, erroneous data, missing input data, or any combination thereof, in the at least one subject-specific sensory metric output.

17. The system of claim 11, wherein the at least one processor is configured to iteratively correct the at least one second dataset collection deficiency by iteratively correcting low quality input data, erroneous data, missing input data, or any combination thereof, in the at least one subject-specific sensor dataset.

18. The system of claim 11, wherein the at least one first SMD comprises:
at least one first SMD sensor to monitor the body of each subject; and
at least one first SMD processor that is configured to execute at least one trained first sensory-monitoring machine learning model;
wherein the at least one trained first sensory-monitoring machine learning model is trained to output the at least one subject-specific sensory metric output for an input of at least one subject-specific first SMD sensor data from the at least one first SMD sensor; and
wherein the at least one processor is configured to interact with the at least one first SMD to receive the at least one subject-specific sensory metric output by inputting the at least one subject-specific first SMD sensor data to the at least one first SMD to receive the at least one subject-specific sensory metric output.

19. The system of claim 11, wherein the at least one first SMD is at least one first pain monitoring device;
wherein the at least one second SMD is at least one second pain monitoring device; and
wherein the at least one subject-specific sensory metric output is a pain metric indicative of a level of pain experienced by each subject during the subject-specific medical procedure.

20. The system of claim 19, wherein the pain metric is a nociception metric.
